(19) United States
(12) Patent Application Publication
(10) Pub. No.: US 2021/0201240 A1
(43) Pub. Date: Jul. 1, 2021

(54) Dynamic Dash Flow For Tracking Key Performance Indicators Of Tasks Associated With Particular Medical Order Types

(71) Applicant: CERNER INNOVATION, INC., Kansas City, KS (US)

(72) Inventors: Vijay Raj Yanamala, Bangalore (IN); Deepak Gupta, Bangalore (IN); Hemalatha Raghavaiah, Bangalore (IN)

(21) Appl. No.: 16/731,213

(22) Filed: Dec. 31, 2019

(51) Int. Cl. G06Q 10/06

(52) U.S. Cl. CPC G06Q 10/06393; G06Q 10/0633; G06Q 10/063112; G06Q 10/063114

(57) ABSTRACT

Systems, methods, and user interfaces provide user-friendly interactive digital workflow graphics for displaying an order's journey through various tasks and key performance indicators (KPIs) for those tasks. The method may include mapping data from electronic medical records (EMR), such as KPIs of various tasks, onto a graphical depiction of a workflow for a particular order type, a particular patient, and/or over a particular period of time. The status of the tasks (e.g., completed, in progress, or queued) may also be visually depicted through color-coding within the interactive digital workflow graphics. The methods here described allow tracking of tasks in a workflow in a way that provides context to the data or KPIs. This not only increases efficiency and assists in streamlining the workflow, but also reduces confusion and documentation errors among different departments and healthcare workers, thereby increasing patient safety.

### BACKGROUND

As is well known, over the last two decades, the use of electronic health record technology has rapidly increased. Hospitals and other healthcare entities rapidly adopted the emerging electronic health record systems because of the advantages they provide over conventional paper-based systems. Moreover, the U.S. Congress spurred the rapid adoption of electronic health record systems by mandating many healthcare entities to switch from conventional paper-based systems to electronic health record systems.

This adoption, however, did not come without challenges. Early electronic systems came with many of the disadvantages and problems that the paper-based systems faced. For example, paper-based systems resulted in patient information being stored at different healthcare facilities and different departments within large healthcare facilities. Likewise, many early electronic health record systems were stored on different media at different healthcare facilities or different departments within a healthcare facility. As a result, efforts have been made to provide ways for medical personnel to quickly and easily review medical records via electronic health records (EHR). Making further use of such EHRs, visual tools such as clinical dashboards can display data, statistics, and key performance indicators (KPIs) using information pulled from EHRs, as illustrated in the prior art dashboard in FIG. 1.

However, documented medical information is often scattered across different data types, and current dashboard solutions generally require users to navigate through different applications, tabs, or sections to get the needed information. Furthermore, dashboards for complicated orders, such as a typical radiology order, can comprise numerous KPIs, making such a dashboard cluttered, confusing, and difficult to utilize quickly and efficiently. For example, as depicted in FIG. 1, a radiology dashboard can give various KPIs, but such dashboard provides no information regarding a radiology order journey and where in a workflow these stats were obtained. Further in complex clinical workflows like radiology where more than one individual (i.e., radiologist, radiology technologist, resident, radiologist transcriptionist) influences the workflow, it is often not possible to standardize an order workflow journey. Thus, interpreting data on a traditional dashboard can be difficult for complex and dynamic workflows or orders, such as in the field of radiology.

### SUMMARY

This summary is intended to introduce a selection of concepts in a simplified form that are further described below in the detailed description section of this disclosure. This summary is not intended to identify key or essential features of the claimed subject matter, nor is it intended to be used as an aid in determining the scope of the claimed subject matter.

At a high level, aspects described herein relate to systems and methods for mapping data from electronic medical records (EMR), such as key performance indicators (KPIs) of various tasks, onto a graphical depiction of a workflow for a particular order type, a particular patient, and/or over a particular period of time. This allows tracking of tasks in a workflow in a way that provides context to the data or KPIs. This not only increases efficiency and assists in streamlining the workflow, but also reduces confusion and documentation errors among different departments and healthcare workers, thereby increasing patient safety.

Additional objects, advantages, and novel features of the technology will be set forth in part in the description which follows, and in part will become apparent to those skilled in the art upon examination of the following or learned by practice of the technology.

### BRIEF DESCRIPTION OF THE DRAWINGS

The present technology is described in detail below with reference to the attached drawing figures, wherein:

FIG. 1 is a traditional prior art dashboard displaying a plurality of key performance indicators and statistics for a radiology order;

FIG. 2 is an example operating environment suitable for use in practice of the technology, in accordance with an aspect described herein;

FIG. 3 is an example computing device suitable for use in practicing embodiments of the technology, in accordance with an aspect described herein;

FIG. 4 is an example user interface having one embodiment of a display comprising a workflow graphic in a multi-order or date-range specific view, in accordance with an aspect described herein;

FIG. 5 is an example user interface having another embodiment of a display comprising a workflow graphic in a single-order view, in accordance with an aspect described herein;

FIG. 6 is an example user interface having another embodiment of a display comprising a workflow graphic in the multi-order or date-range specific view and also depicting a plurality of blocked workflow tasks, in accordance with an aspect described herein;

FIG. 7 is an example user interface having another embodiment of a display comprising a workflow graphic in the multi-order or date-range specific view with all order tasks depicted as completed, in accordance with an aspect described herein;

FIG. 8 is an example user interface having another embodiment of a display comprising a workflow graphic in the single-order view and depicting vetting added to the workflow, in accordance with an aspect described herein;

FIG. 9 is an example user interface having another embodiment of a display comprising a workflow graphic in the single-order view depicting completion of the vetting and queuing the order to a radiologist for dictation, in accordance with an aspect described herein;

FIG. 10 is an example user interface having another embodiment of a display comprising a workflow graphic in the single-order view depicting completion of vetting, dictation, and transcription, with the order queued to be signed out, in accordance with an aspect described herein;

FIG. 11 is an example user interface having another embodiment of a display comprising a workflow graphic in the single-order view depicting a vetted order that is finalized and signed out, in accordance with an aspect described herein;

FIG. 12 is a flow chart of a method of displaying updated process flow data in an interactive digital workflow graphic, in accordance with an aspect described herein;

FIG. 13 is a flow chart of a method of building a workflow graphic for displaying updated process flow data, in accordance with an aspect described herein;

FIG. 14 is a flow chart of a method of hospital management using the workflow graphic to unblock blockers identified in a period of time, in accordance with an aspect described herein;

FIG. 15 is a flow chart of a method of radiation personal using the workflow graphic to visualize an order's journey through the workflow, in accordance with an aspect described herein; and

FIG. 16 is a flow chart of a method of hospital management using the workflow graphic to visualize key performance indicators (KPI), in accordance with an aspect described herein.

The drawing figures do not limit the present technology to the specific embodiments disclosed and described herein. The drawings are not necessarily to scale, emphasis instead being placed upon clearly illustrating the principles of the invention.

### DETAILED DESCRIPTION

The subject matter of the present technology is described with specificity herein to meet statutory requirements. However, the description itself is not intended to limit the scope of this disclosure. Rather, the inventors have contemplated that the claimed or disclosed subject matter might also be embodied in other ways, to include different steps or combinations of steps similar to the ones described in this document, in conjunction with other present or future technologies. Moreover, although the terms “step” and/or “block” might be used herein to connote different elements of methods employed, the terms should not be interpreted as implying any particular order among or between various steps herein disclosed unless and except when the order of individual steps is explicitly stated.

With regard to the drawings in general, we show certain items in block-diagram form more for being able to reference something consistent with the nature of a patent rather than to imply that a certain component is or is not part of a certain device. Similarly, although some items are depicted in the singular form, plural items are contemplated as well (e.g., what is shown as one data store might really be multiple data stores distributed across multiple locations). But showing every variation of each item might obscure some embodiments. Thus for readability, we show and reference items in the singular (while fully contemplating, where applicable, the plural).

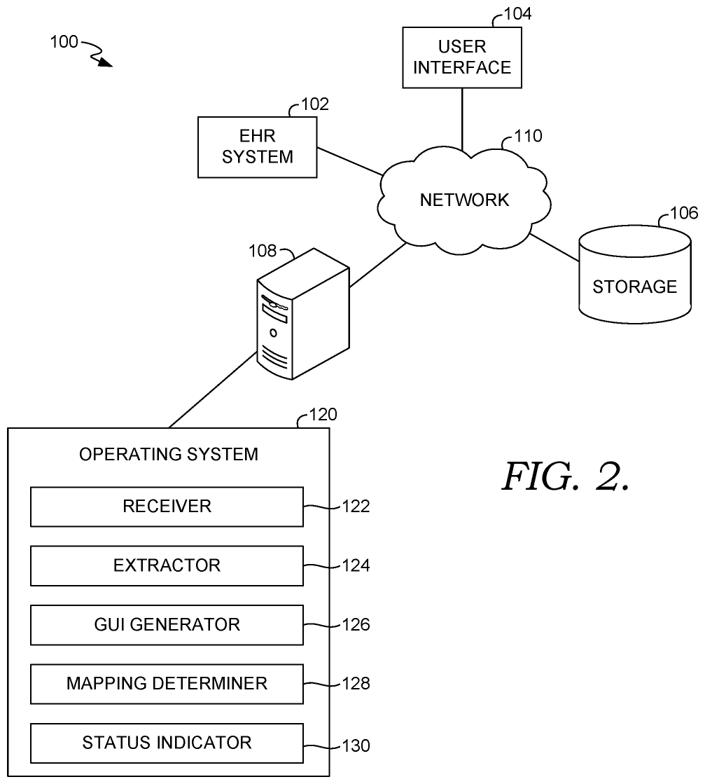

Turning now to FIG. 2, an example operating environment 100 for providing visual information that increases workflow efficiency is illustrated. As illustrated in FIG. 2, the example operating environment 100 includes an electronic health record (EHR) system 102, a user interface 104, a data storage 106, and a computing device 108. Each of these elements is shown communicating through the network 110. The computing device 108 is shown including an operating system 120. The operating system 120, as shown in FIG. 2, includes a receiver 122, an extractor 124, a GUI generator 126, a mapping determiner 128, and a status indicator 130. Each of these elements will be described in more detail below.

As noted above, each of these components may communicate through the network 110. The network 110 may include, without limitation, one or more local area networks (LANs) and/or wide area networks (WANs). In example implementations, the network 110 comprises the Internet and/or a cellular network, amongst any of a variety of possible public and/or private networks. Other wireless technologies for transferring data, such as Bluetooth, are contemplated as well. Though components of the operating environment 100 are shown communicating through the network 110, other arrangements are contemplated. For example, one or more of the components of the operating environment 100 may be in direct communication via a communication bus.

It will be understood that the operating environment 100 is provided only as an example of one embodiment suitable for practicing the technology. Other arrangements of elements (e.g., components, machines, interfaces, functions, orders, groupings of functions, etc.) can be used in addition to or instead of those shown, while some may be omitted from FIG. 2 for the sake of clarity in describing the technology.

The EHR system 102 may include one or more data stores of health records, which may be included in the storage 106, and may further include one or more computers or servers that facilitate the storing and retrieval of the health records. Although the storage 106 is depicted as a single data store component, the storage 106 may be embodied as one or more data stores or may be in the cloud. In some embodiments, the EHR system 102 may be implemented as a cloud-based platform or may be distributed across multiple physical locations and may be the same as or distinct from the storage 106. The EHR system 102 may further include record systems, which store real-time or near real-time patient (or user) information, such as wearable, bedside, or in-home patient monitors, for example, and may store patient EHRs. For example, the EHR system 102 may comprise one or a plurality of EHR systems such as hospital EHR systems, health information exchange EHR systems, ambulatory clinic EHR systems, or other systems having health-related records for one or more patients, and may be implemented in the computing device 108.

Generally, EHRs, sometimes referred to as electronic medical records (EMRs), may comprise electronic clinical documents such as images, clinical notes, orders, summaries, reports, analyses, dictations, transcriptions, information received from clinical applications and medical devices, or other types of electronic medical documentation relevant to a particular patient's condition and/or treatment. Electronic clinical documents may contain various types of information relevant to the condition and/or treatment of a particular patient and can include information relating to, for example, patient identification information, images (e.g., radiology imaging and x-rays), audio or video recordings, alert history, culture results, physical examinations, vital signs, past medical histories, surgical histories, family histories, histories of present illnesses, current and past medications, allergies, symptoms, past orders, completed orders, pending orders, tasks, transcripts, lab results, other test results, patient encounters and/or visits, immunizations, physician comments, nurse comments, other caretaker comments, clinician assignments, and a host of other relevant clinical information. The content and volume of such information in an EHR system are not intended to limit the scope of the present disclosure in any way.

The user interface 104 is also illustrated in the operating environment 100. An embodiment of the user interface 104 takes the form of a user interface operated by a software application or set of applications on a client computing device such as a personal computer, laptop, smartphone, or tablet computing device. In an embodiment, the application is a Web-based application or applet. Embodiments of the user interface 104 may facilitate accessing, receiving, and communicating information from a user or healthcare provider about a specific patient or population of patients, such as patient history; healthcare resource data; variables measurements, time series, and predictions (including plotting or displaying the determined outcome and/or issuing an alert); or other health-related information, and facilitates the display of results, recommendations, or orders. For example, the user interface 104 may communicate with the EHR system 102 through the network 110 to receive patient EMRs or any other information stored in the EHR system 102. In another example, the user interface 104 may communicate with sensors or other electronic input devices to receive any real-time or near real-time patient information.

In some embodiments, the user interface 104 may include a GUI for conveying visual information. The user interface 104 may take the form of a computer monitor, which may be in communication with a server to receive information and visually convey it using the GUI. For example, computer monitors may include stand-alone monitors that are independent of the processor and are in communication with the processor through a communication bus or through wireless technology. Such computer monitors may generally be any shape and size.

In some embodiments, the user interface 104 may be integrated with one or more other computer components, such as storage hardware or processors. For example, such computer systems may include laptops, tablets, smartphones, and the like. These systems may be considered mobile devices. User interfaces associated with mobile devices may be smaller than the computer monitors previously described. Some special mobile devices may have user interfaces that are greater than standard monitor sizes, while others may have user interfaces less than standard mobile devices. These special mobile devices are also contemplated within the scope of and may also be suitable for use with the present technology. Mobile devices may communicate with other technologies, such as other components of FIG. 2, through wireless capabilities, such as using the network 110.

In some aspects, the user interface 104 may be touch sensitive. In general, a touch sensitive user interface may receive a user input at the user interface 104. In some cases, the input received will be determined based on the visual information presented by the GUI of the user interface. For example, touch sensitive user interfaces may detect an input based on sensing electrical conductivity of another object, such as a finger or stylus; based on pressure applied to the user interface; or by determining a position of an object and associating its position with a location of the user interface 104. These types of touch sensitive user interfaces and others are known in the art and will not be described in detail here for the sake of clarity; however, they are contemplated for use in the present technology.

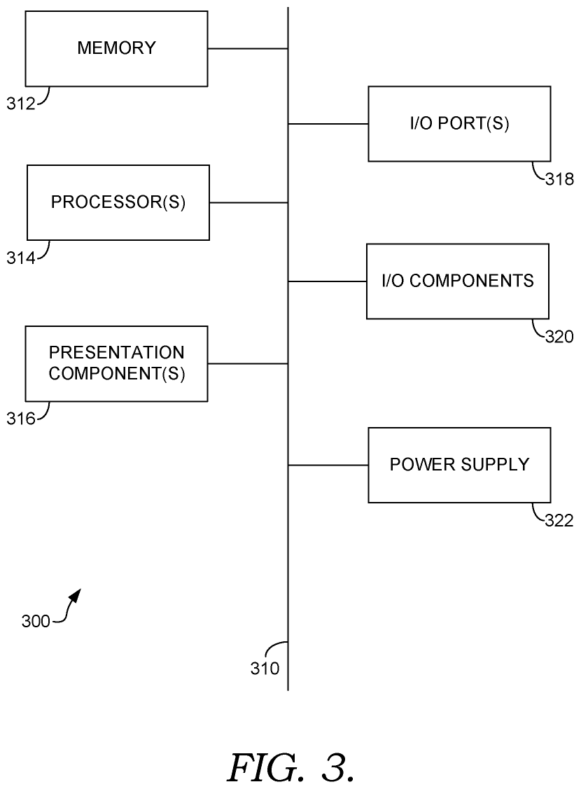

As illustrated in FIG. 2, the operating environment also includes the computing device 108. The computing device 108 generally comprises one or more processors operable to receive instructions and process them accordingly, and may be embodied as a single computing device or multiple computing devices communicatively coupled to each other. For example, computing device 108 may process computer-usable instructions stored on computer-readable media. In one embodiment, processing actions performed by computing device 108 are distributed among multiple locations, such as one or more local clients and one or more remote servers, and may be distributed across the other components of the operating environment 100. In an embodiment, the computing device 108 comprises one or more computing devices, such as a server, desktop computer, laptop, or tablet, cloud-computing device or distributed computing architecture, a portable computing device such as a laptop, tablet, ultra-mobile P.C., or a mobile phone. One example of the computing device 108 suitable for use in practice of the current technology is depicted in FIG. 3, as later described herein.

With continued reference to FIG. 2, some embodiments of the computing device 108 include a computer software stack, which in some embodiments operates in the cloud, as a distributed system on a virtualization layer within computing device 108, and may include the operating system 120. Some embodiments of the operating system 120 comprise a distributed adaptive agent operating system. The operating system 120 may be implemented as a platform in the cloud, which is capable of hosting a number of services that may run as a local or distributed stack in the cloud, on one or more personal computers or servers. For example, as illustrated in FIG. 2, the operating system 120 may host the receiver 122, the extractor 124, the GUI generator 126, the mapping determiner 128, and the status indicator 130.

In some cases, services hosted by the operating system 120 are associated with one or more virtual assistant applications, services, or routines. For example, such applications, services, or routines may operate on one or more user devices and servers, such as the computing device 108, and may be shared or distributed across one or more user devices and servers, one or more other components, or be implemented in the cloud. Moreover, the functions performed, or services carried out by these functions, may be implemented at appropriate abstraction layer(s) such as the operating system layer, application layer, hardware layer, and the like of the computing system(s).

In some cases, the functions and/or the embodiments described herein can be performed, at least in part, by one or more logic components, which may be stored in the data storage 106. For example, and without limitation, illustrative types of logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. In general, the logic comprises rules, conditions, associations, classification or prediction models, pattern inference algorithms, or other criteria utilized by the operating system 120 to execute services. In some embodiments, the logic may utilize pattern recognition, fuzzy logic, neural network, finite-state machine, support vector machine, logistic regression, clustering, or machine learning techniques, similar statistical classification processes, or combinations of these processes.

An example of the computing device 108 is provided in FIG. 3. Computing device 108 is one example of a suitable computing environment for practicing the technology, but is not intended to suggest any limitation as to the scope of use or functionality of the present technology. In some cases, the technology may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to code that perform particular tasks or implement particular abstract data types. The technology may be practiced in a variety of system configurations, including hand-held devices, consumer electronics, general-purpose computers, more specialty computing devices, etc. The technology may also be practiced in distributed computing environments where tasks are performed by remote-processing devices that are linked through a communications network.

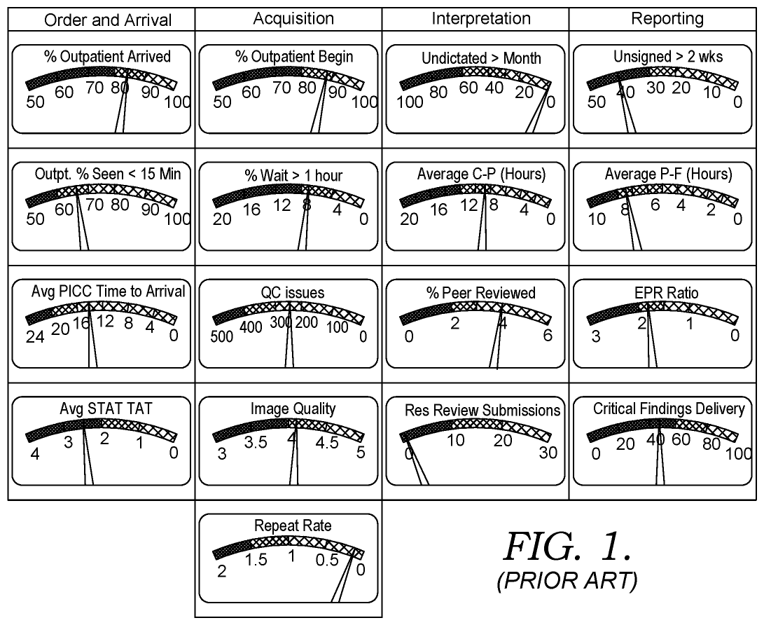

With reference to FIG. 3, computing device 108 includes bus 310 that directly or indirectly couples the following devices: memory 312, one or more processors 314, one or more presentation components 316, input/output (I/O) ports 318, input/output components 320, and illustrative power supply 322. The bus 310 represents what may be one or more busses (such as an address bus, data bus, or combination thereof). Although the various blocks of FIG. 3 are shown with lines for the sake of clarity, in reality, delineating various components is not so clear, and metaphorically, the lines would more accurately be gray and fuzzy. For example, one may consider a presentation component such as a display device to be an I/O component. Also, processors have memory. The inventors recognize that such is the nature of the art, and reiterate that the diagram of FIG. 3 is merely illustrative of an exemplary computing device that can be used in connection with one or more embodiments of the present disclosure. Distinction is not made between such categories as “workstation,” “server,” “laptop,” “hand-held device,” etc., as all are contemplated within the scope of FIG. 3 and reference to “computing device,” such as the computing device 108 of FIG. 1.

With reference again to FIG. 3, the computing device 108 typically includes a variety of computer-readable media. Computer-readable media can be any available media that can be accessed by the computing device 108 and includes both volatile and nonvolatile media, removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the computing device 108. Computer storage media does not comprise signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The memory 312 includes computer storage media in the form of volatile and/or nonvolatile memory. The memory 312 may be removable, non-removable, or a combination thereof. Exemplary hardware devices include solid-state memory, hard drives, optical-disc drives, etc. The computing device 108 includes one or more processors that read data from various entities such as the memory 312 or I/O components 320. The presentation component(s) 316 present data indications to a user or other device. Exemplary presentation components include a display device, speaker, printing component, vibrating component, the user interface 104, etc.

The I/O ports 318 allow computing device 108 to be logically coupled to other devices including the I/O components 320, some of which may be built in. Illustrative components include a microphone, joystick, game pad, satellite dish, scanner, printer, wireless device, etc. The I/O components 320 may provide a natural user interface (NUI) that processes air gestures, voice, or other physiological inputs generated by a user. In some instance, inputs may be transmitted to an appropriate network element for further processing. A NUI may implement any combination of speech recognition, touch and stylus recognition, facial recognition, biometric recognition, gesture recognition both on screen and adjacent to the screen, air gestures, head and eye-tracking, and touch recognition associated with displays on the computing device 108. The computing device 108 may be equipped with depth cameras, such as, stereoscopic camera systems, infrared camera systems, RGB camera systems, and combinations of these for gesture detection and recognition. Additionally, the computing device 108 may be equipped with accelerometers or gyroscopes that enable detection of motion.

As previously noted and illustrated in FIG. 2, an embodiment of the operating system 120, operable to run on the computing device 108, may comprise the receiver 122, the extractor 124, the GUI generator 126, the mapping determiner 128, and the status indicator 130. Specifically, the operating system 120 may be a software application or computer program with code segments or modules configured to be executed by the computing device 108 to perform the functions or methods described herein. The code segments or modules may include the receiver 122, the extractor 124, the GUI generator 126, the mapping determiner 128, and the status indicator 130, in addition to any other code segments or modules for facilitating the methods and functions described herein.

In general, the receiver 122 receives information. In some aspects, the receiver 122 receives stored patient information, such as EHRs received from the EHR system 102. In some aspects, the receiver 122 may receive clinical documents or information as an input, for example, a lab assistant, nurse, or document specialist inputting patient information via an input associated with the computing device 108 and/or via the user interface 104. In some embodiments, the receiver 122 may also receive real time or near real time patient information, such as measured physiological parameters from sensors or the user interface 104. In some embodiments, the receiver 122 may receive order information and/or patient information. The receiver 122 may also store this order or patient information temporarily or permanently, such as in the storage 106.

Patient information may include historical clinical diagnoses, such as cancer, depression, hypothyroidism, diabetes, pregnancy, and the like. Other examples of patient information include medications taken by the patient and/or prescribed to the patient. More examples of patient information include stored measurements related to various physiological parameters, such as weight, body mass index (BMI), thyroxine (T4), Thyroglobulin (Tg), and thyrotropic hormone (TSH), and the like. Patient information may also include historical physiological measurements associated with the physiological parameters and determined at different times.

Order information may include information related to tasks for completion of a particular order or order type, such as key performance indicators (KPIs). The tasks may include: patient registration, placement of orders, examination of orders, filling of orders, scheduling of procedures, conducting an exam which may include testing or imaging of a patient, storing or printing of imaging or testing data, transmitting imaging or testing data to medical staff responsible for analysis or diagnosis, analyzing imaging or testing data by medical staff, reporting on analysis by medical staff based on the imaging or testing data, and transmitting of diagnosis report to patient and/or treating physician. The KPIs may include statistics or data regarding completion or turnaround time for any of the tasks, documentation of critical task-related information, task outcomes, task statistics, task comparisons, and task projections or predictions. More specifically, the KPIs may include any of the following statistics for a single patient, for a single order, and/or on average for a plurality of patients or orders: the amount of time a task takes to complete, patient arrival times (e.g., timeliness to an appointment), amount of time for a patient to be seen, image quality, repeat rates, percentage peer reviewed, turnaround time for transcribing, turnaround time for dictating, turnaround time for reporting results, turnaround time for signing a report, tracking information regarding documentation of critical information such as radiation dose and technical comments, and any other key performance indicators tracked by the medical facility for a given task. While only a few examples of tasks and related KPIs have been included in this description as examples, it will be recognized that these are only a few of many. Other examples are not included for the sake of brevity, but are intended to be within the scope of this disclosure.

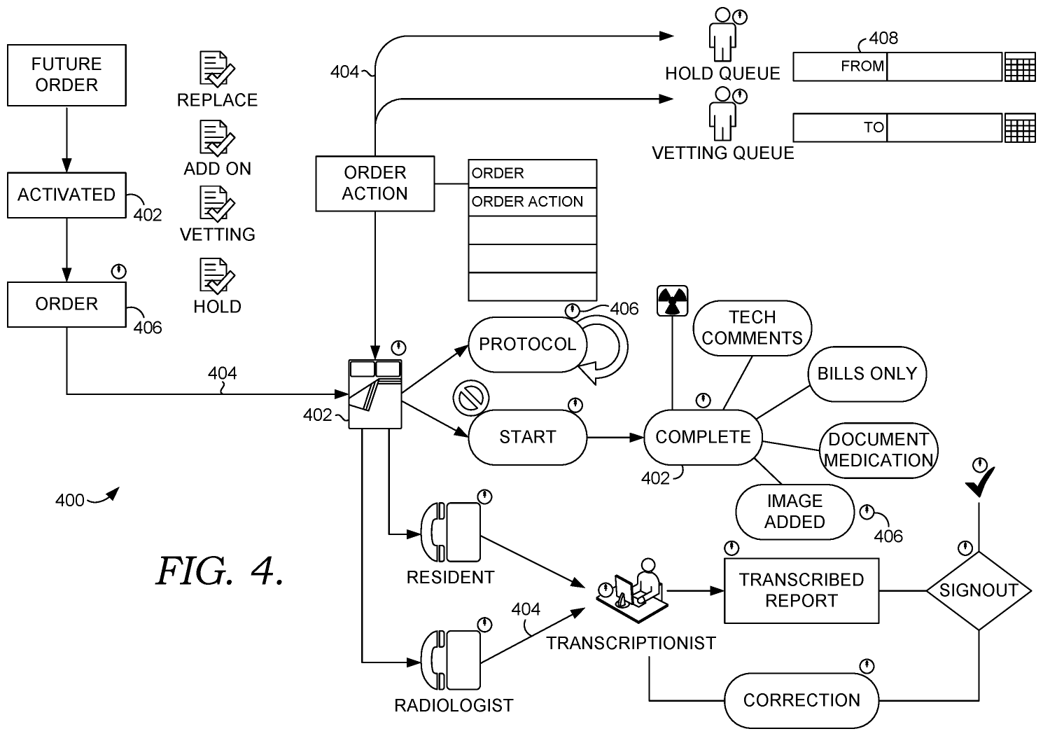

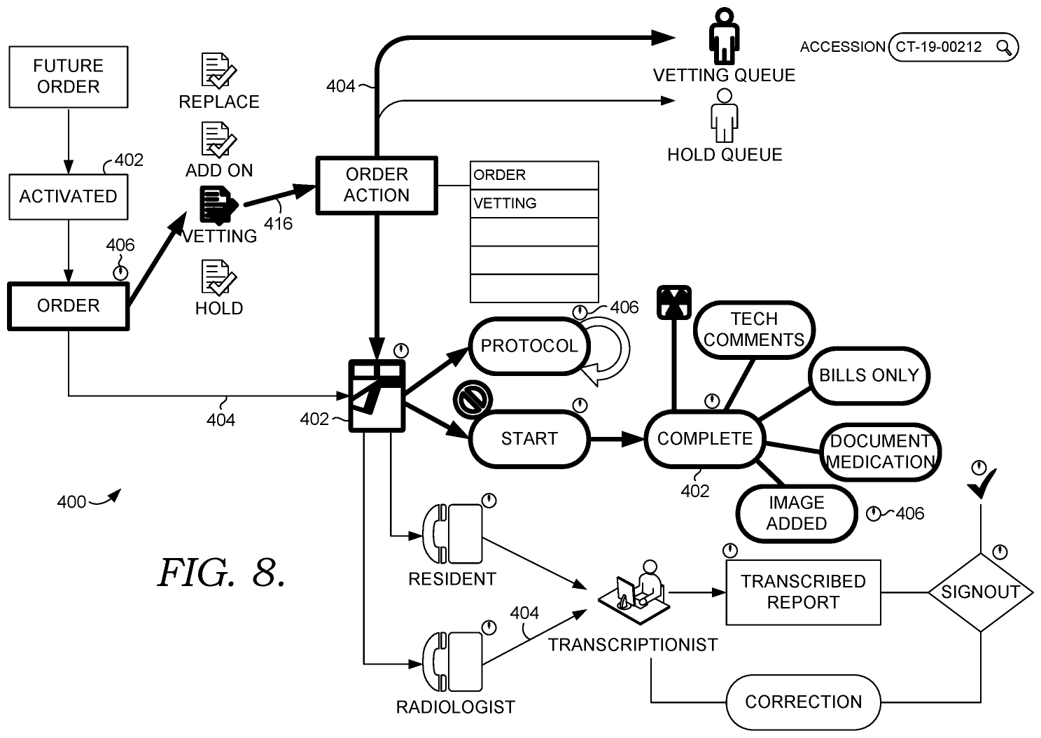

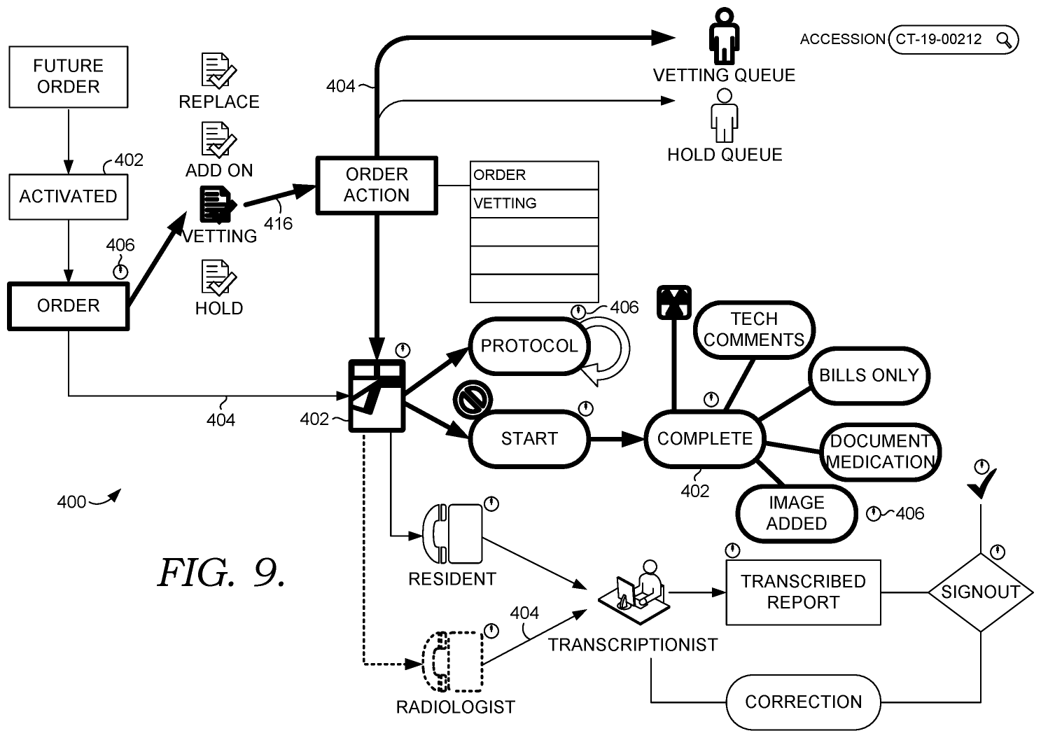

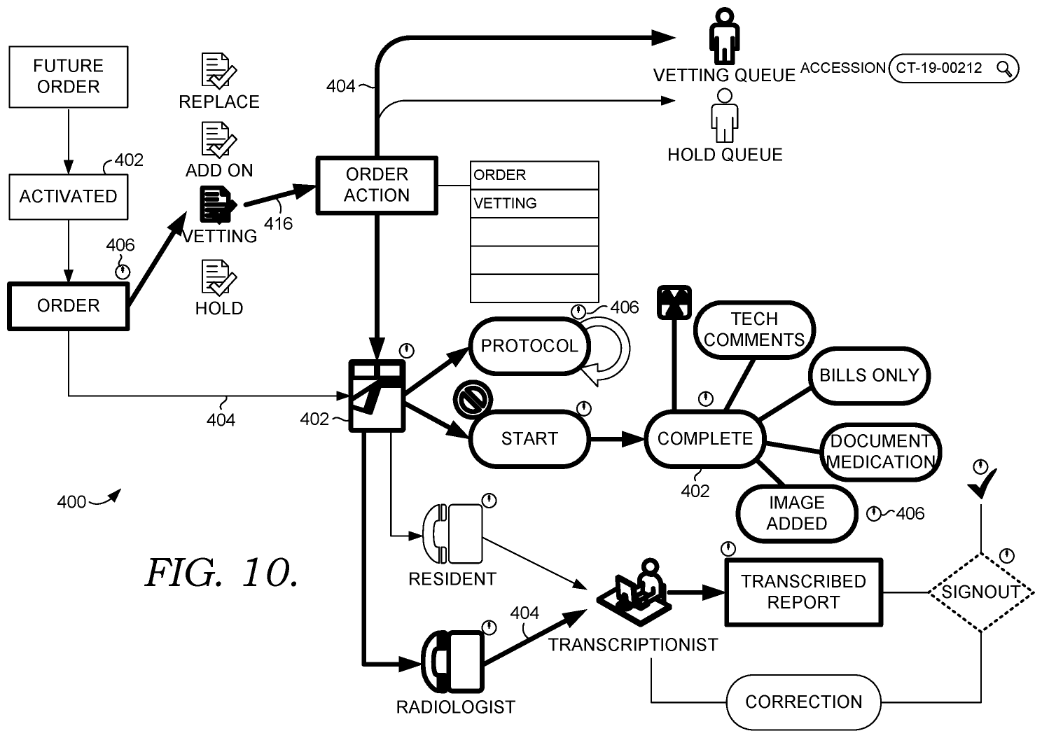

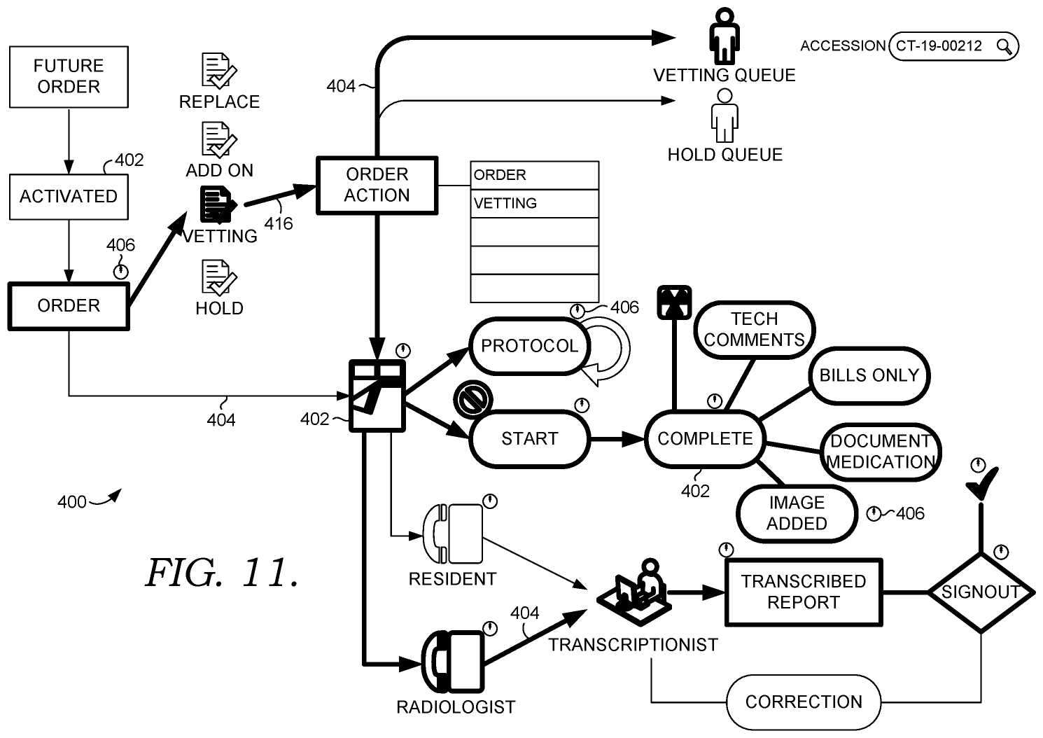

In one specific example, as depicted in FIGS. 4-11, tasks associated with an order for a radiological exam may include order related events or tasks which happen before a technologist starts the exam, such as: isolation procedures, placing an order, placing a future order, activating a future order at the desired time or date, replacing an order action, adding on to the order or an order action, vetting the order action (by a radiologist or a non-radiologist), and holding the order or an order action (by a radiologist or a non-radiologist). The tasks associated with the radiological exam may also include order related events or tasks specifically regarding a technologist's workflow, such as: conducting exam protocol, starting the exam, completing the exam, recording the radiation dose used, adding technical comments to the corresponding record, adding billing information to the corresponding record, documenting medication, and conducting image management routines. Furthermore, the tasks associated with the radiological exam may also include report-related events or tasks, such as: dictation of report by resident or radiologist, transcribing of report, signing-out of the report, correcting of the report, and signing/finalizing the report.

The patient and order information received by the receiver 122 may also include, for example, a time stamp. For example, the order information stored in the EHR may be associated with time stamps, including when particular tasks within a workflow of a given order or order type are received and/or completed. Tasks may also be associated with time stamps when there are changes in the order regarding a particular task. In some cases, the time stamp may represent the time that the patient information was stored. In addition to or alternatively, the time stamp may represent the time that the patient information was collected. Furthermore, medication information may be associated with one or more start dates and end dates, which may also include time stamps for change in dosage or frequency of taking the medication. Another example of patient information having associated time stamps includes clinical diagnoses, which may also include one or more onset dates and termination dates.

The extractor 124 generally parses and extracts information from the raw patient or task-related information received by the receiver 122, and stores the extracted information in a useable format. For example, raw patient information may be received from and EHR stored on the storage 106. The extractor 124 may extract information within the EHR such as patient demographics, diagnoses information, medication information, and measured physiological parameter values. Additionally or alternatively, the extractor 124 may extract information regarding KPIs associated with various tasks for a particular order or particular type of order. Parsing the information may include determining the time stamp associated with each of the entries for the diagnoses, medication information, task completion, and the like, such as a time that a particular task or lab test was performed.

Continuing, parsing the received patient information may include determining date stamps from metadata associated with an entry. For example, a physician may have entered a particular diagnosis into the patient's EHR. The date the entry was input may be associated as metadata, which when parsed, may be included as an onset date for the diagnosis if one was not expressly input by the physician. Additionally or alternatively, parsing may include determining a therapeutic or drug class for a medication. Furthermore, imaging data may include a timestamp in metadata from when the image was taken. The extractor 124 may be configured to parse different file types or files having different forms and formats for storing KPIs, timestamps, and associated metadata.

Once parsed, in some embodiments, the extractor 124 may store the information in a useable format. For example, workflow tasks related to a particular order or order type and their statuses (e.g., completed, in progress, queued) may be stored corresponding to order types, start dates and times, end dates and times, and various KPIs associated with particular ones of the tasks. It will be appreciated that other KPIs and workflow tasks performed to obtain such information may be used in various order workflows. However, for the sake of brevity and to not obscure the disclosure of the present technology, not all KPI and workflow task information can be described. Thus, while certain patient information, KPIs, and workflow tasks may not be expressly described herein, it is contemplated by the inventors that receiver 122 may receive any electronically stored or real time patient information, while it is also contemplated that extractor 124 may parse any of the information from receiver 122, extract the parsed information, and temporarily or permanently store the parsed information in a usable format. Furthermore, in some embodiments, the data may be received in a usable format and the extractor may be omitted without departing from the scope of the technology described herein.

The GUI generator 126 generally dictates how to visually present an interactive digital workflow graphic 400 and KPIs associated with tasks on the workflow graphic 400 in a manner that provides context to the KPIs and may communicate the data for display by the user interface 104. FIGS. 4-11 illustrate various embodiments of visually communicated information on a user interface, such as user interface 104, as generated by GUI generator 126. FIGS. 4-11 will be referenced through the discussion of the GUI generator 126; however, these embodiments are by no means meant the limit the possible visual representations of data that can be generated by the GUI generator 126 that still provide an interactive digital workflow graphic and KPIs associated with various tasks thereon, as they are only examples.

Specifically, the GUI generator 126 can access from any database described herein (such as the storage 106) the interactive digital workflow graphic 400 having graphical depictions of a plurality of tasks 402 to be completed for a particular medical order type, a particular patient, and/or a particular patient's particular medical order. In some embodiments, the interactive digital workflow graphic 400 may be one of a plurality of interactive digital workflow graphics for each of a plurality of order types. At least some of the tasks for the order can be graphically connected by linking graphics 404 indicating a required or desired completion sequence of the tasks 402 displayed in the workflow graphic 400, as depicted in FIGS. 4-11. In some embodiments, the workflow graphic 400 is built based on user selection of a particular medical order type, facilities or departments involved with the plurality of tasks for the particular medical order type, and/or sequences of the plurality of tasks within and between each of the facilities or departments for the particular medical order type. In some embodiments, the workflow graphic 400, its graphical depiction of the tasks 402, and its linking graphics 404 may be pre-designed and saved in a database, such as storage 106, and then accessed via the GUI generator 126. Additionally or alternatively, various aspects of the workflow graphic 400 can be included or excluded based on user selections or other filtering options described herein.

The mapping determiner 128 can use the information received from the receiver 122 and/or parsed by the extractor 124 to determine which content or data to link to dashboard elements 406 visually displayed on the workflow graphic 400. The dashboard elements 406, as depicted in FIGS. 4-11, may be graphical elements, such as a timer icon that traces the turnaround time and displays when hovered over by a mouse curser, for example. However, any graphical element or text may be used for the dashboard elements 406. Furthermore, the mapping determiner 128 can map KPIs from the EHR System 102 to the dashboard elements 406 to be depicted on or adjacent to at least some of the graphical depictions of the tasks 402. In some embodiments, the KPIs are mapped to be displayed directly on the dashboard elements 406 (e.g., a numerical value or percentage displayed next to the graphical depiction of the task 402) or are mapped to be displayed or overlaid onto the workflow graphic 400 in response to a cursor hovering over or selecting one of the dashboard elements 406.

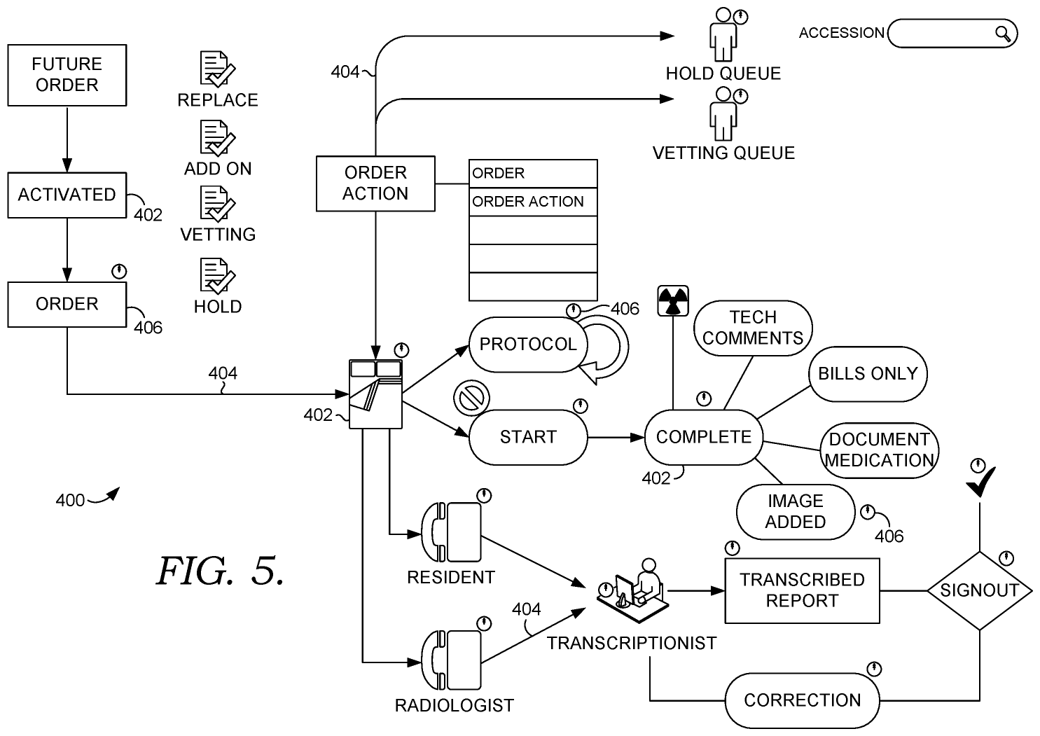

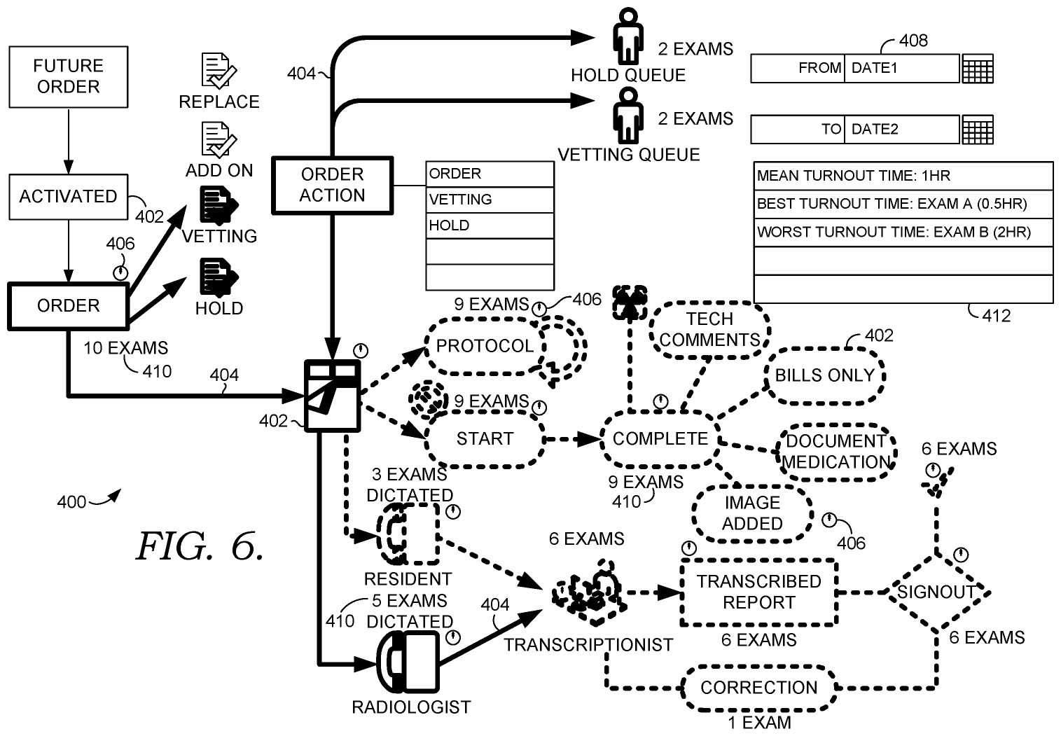

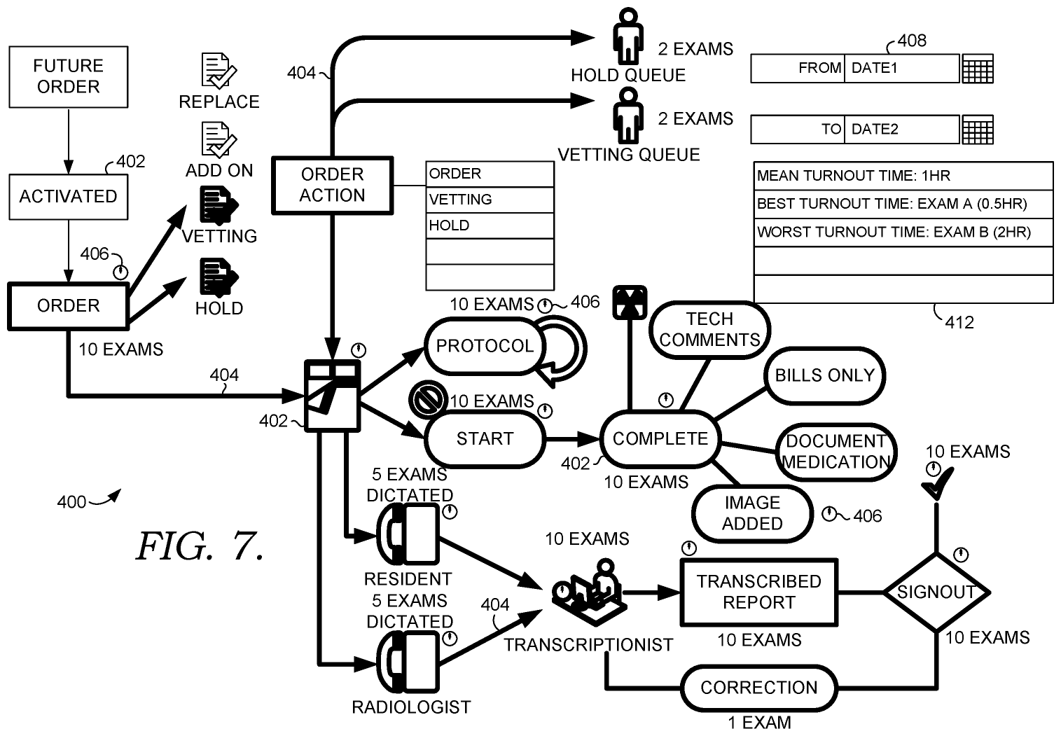

The mapping determiner 128 may map data to the dashboard elements 406 based on user-entered selections, such as filtering options described herein. Specifically, the interactive digital workflow graphic 400 can include selectable view options, such as an option to display a single-order view in which data or KPIs for a single patient and a single order is displayed as illustrated in FIG. 5, or an option to display a multi-order view with selectable filtering options as illustrated in FIG. 4 to determine which orders of the particular type to include, such as date selection options 408. For example, a user may select a date range such that KPIs (or averages or statistics related to KPIs for various ones of the tasks) for the orders of the selected type that were made during that date range are mapped to the dashboard elements 406. Text labels 410 indicating how many of the plurality of orders during the selected date range have been completed for each of the tasks may also be added to the workflow graphic 400, as illustrated in FIGS. 6 and 7. Other visual indicators on the workflow graphic 400 may provide KPIs or statistical data for the workflow overall, as depicted with graphic 412, such as a mean turnaround time for all task, a particular exam or order with the best turnaround time for the selected time period, and a particular exam or order with the worst turnaround time for the selected period of time, as also illustrated in FIGS. 6 and 7.

Furthermore, in some embodiments, the GUI generator 126 or the mapping determiner 128 may add additional linking graphics 414 to the workflow graphic 400 based on certain completed tasks, particular metadata, or user selections. For example, if at least some of the orders of a selected order type within a selected date range were flagged for vetting, included an add on, were replaced with another exam, or were being held for various reasons, as depicted in FIGS. 6-11, the GUI generator 126 or the mapping determiner 128 may add the additional linking graphics 414 between relevant ones of the tasks 402 in response to any resulting additional workflow tasks.

The status determiner 130 may use data from the EHR system 102 or any other received status information to visually indicate which of the plurality of tasks is completed, is in progress, or is queued for a pre-defined threshold number or percentage of orders of the particular medical order type, as depicted in FIGS. 6-11 (with different cross-hatching representing different colors). Visually indicating a status may be performed using color-coding, different shapes, different symbols, different patterns, text labels, or any other methods for visually differentiating graphic elements. For example, graphical depictions of particular ones of the tasks that are completed may be highlighted in green and/or the linking graphics 404 leading to the completed task may be highlighted or changed to green. Likewise, the graphical depiction of particular ones of the tasks that are in progress may be highlighted in blue and/or the linking graphics 404 leading to the in progress task may be highlighted or changed to blue. Similarly, the graphical depiction of particular ones of the tasks that are queued may be highlighted in red and/or the linking graphics 404 leading to the in progress task may be highlighted or changed to red. In some embodiments, when the multi-order view is selected, blocked events may be depicted in red while completed events may be depicted in green. Note that these color combinations are merely exemplary and do not limit the scope of colors or other visual indicators that can be used by the status determiner 130. Blocked events or tasks, as used herein, refers to a task taking more than a threshold amount of time for completion (or never being completed at all) and/or a threshold number of orders of a particular order type taking more than the threshold amount of time for completion of that particular task. For example, in some embodiments, a blocked event may be depicted (e.g., shown in red) even if only one of a plurality of orders has not completed a particular required task.

In some embodiments, the status determiner 130 can mark one of the tasks 402 as completed based on receipt of a particular type of file or a particular type of data, based on user indication of completion such as with a signature or digital signature, or based on indication that a next task was started, particularly if the next task cannot proceed without the previous task. In some embodiments, any of the tasks 402 may be considered queued when a previous one of the tasks 402 is indicated complete and may be considered to be in progress when a start time is provided but no completion time or other indication of completion of the task is received.

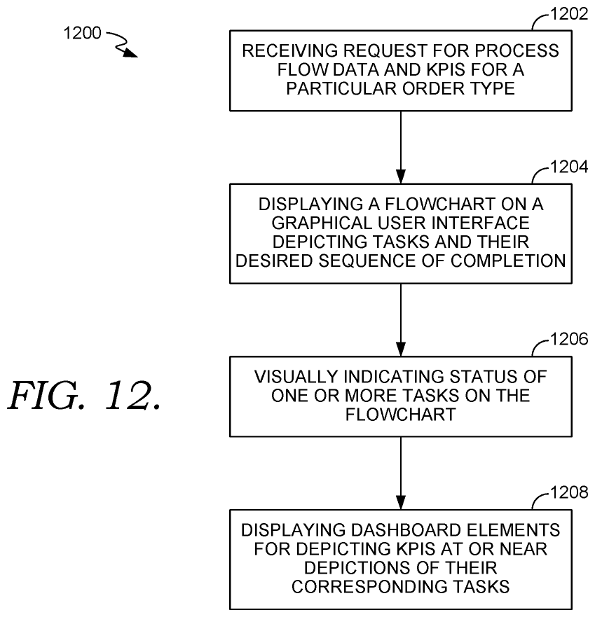

With reference first to FIG. 12, one method 1200 of displaying updated process flow data in the systems described herein may include the steps of: receiving a request for process flow data and KPIs, as depicted in block 1202; displaying a flow chart, such as the interactive digital workflow graphic 400, on a graphical user interface as depicted in block 1204; visually indicating which of tasks are completed, in progress, or queued, as depicted in block 1206; and displaying on the flow chart dashboard elements 406 on or adjacent to depictions of the tasks, as depicted in block 1208.

The step 1202 of receiving a request for process flow data and KPIs may include a request for a particular order type within the EHR system 102 or other EHR databases at a particular medical facility over a selected period of time or for otherwise filtered information. For example, the particular order type may be a radiological exam, and the selectable order options may include replacing an order, adding onto an order, vetting an order, or holding an order. In some embodiments, the request may be for a specific order for a specific patient, to visually depict that order's workflow. The flow chart displayed in step 1204 may include any of the visual components noted above for the interactive digital workflow graphic 400, including depictions of the tasks 402 associated with a particular order type or a particular order, the linking graphics 404, and the dashboard elements 406 as described herein.

The linking graphics 404 may be arranged for visually indicating a required or desired sequence of completion of the tasks. In some embodiments, step 1204 of the method may further include displaying additional linking graphics 414 on the flow chart connecting additional tasks when a particular one of the selectable order options are selected by a user. For example, compare FIG. 5 to FIGS. 8-11, which depict additional linking graphics to a vetting step added to the workflow. Furthermore, in some embodiments, the method may further include providing a date selection element 406 and receiving filtering instructions therefrom, such that the KPIs to be displayed in the flowchart are filtered based on a selected period of time or dates entered into the date selection element, as illustrated in FIGS. 6-7. For example, radiology orders at a particular facility during the past two days may be displayed, with text labels indicating how many of each of those orders made it to each particular task and/or were seen through to completion for each particular task. Additionally or alternatively, as depicted in FIGS. 5 and 8-11, the method may include providing a selectable individual order view where, when the selectable individual order view is selected, the KPIs are filtered to display only ones of the KPIs associated with a single patient or a single order of the particular order type.

Visually depicting the status of the tasks, as in step 1206, may include the color-coding described herein or other various methods. In some embodiments, where multiple orders are displayed on the flow chart, the statuses may be indicated for a pre-defined threshold number or percentage of orders of a particular order type within a selected time period. For example, a visual indication that a particular one of the tasks is completed may require 100% of the orders of that order type over the given date or time range to be completed. Likewise, a color indicating a blockage at a particular task may require only one of the orders to be stalled at a particular one of the tasks. Other thresholds may be used without departing from the scope of the technology described herein.

The dashboard elements 406 in step 1208 may depict the KPIs for at least one of the tasks displayed in the flow chart, either automatically on the dashboard elements 406 or overlaid on the flow chart when the dashboard element 406 is hovered over or selected by a cursor or other user interface selection tools. As earlier noted herein, the KPIs may include statistics or data regarding any of the following: completion or turnaround time for any of the tasks, documentation of critical task-related information, task outcomes, task statistics, task comparisons, and task projections or predictions.

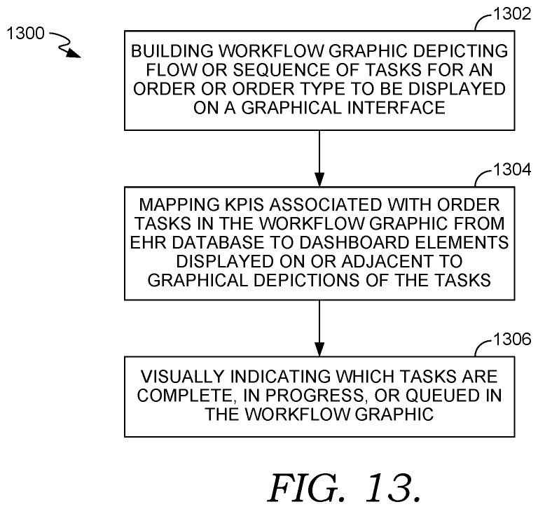

With reference to FIG. 13, a method 1300 of building a workflow graphic, such as the interactive digital workflow graphic 400, may be implemented on any of the computer systems described herein and may comprise the steps of: building a workflow graphic depicting a flow or sequence of a plurality of tasks for a particular order or order type to be displayed on a graphical user interface, as depicted in block 1302; mapping KPIs for tasks in the workflow graphic from an EHR to dashboard elements 406 on or adjacent to graphical depictions of the tasks, as depicted in block 1304; and visually indicating which of the tasks is completed, in progress, or queued within the workflow graphic, as depicted in block 1306.

As noted above, the workflow graphic may be the interactive digital workflow graphic 400 described herein and may similarly include graphical depictions of tasks to be completed for a particular medical order type. Graphical depictions of the tasks may also be graphically connected by linking graphics indicating a required or desired completion sequence of the tasks. Furthermore, the workflow graphic 400 may be built based on the particular medical order type, facilities or departments involved with the order tasks, and sequences of the tasks within and between each of the facilities or departments for the particular medical order type.

The mapping of KPIs may be performed by indicating digital locations and/or file types of relevant electronic health records (EHRs) to be accessed by the workflow graphic 400 and determining which information within the EHRs should be linked to the dashboard elements 406. As in previous embodiments, the KPIs may be mapped to be displayed directly on the dashboard elements 406 or may be mapped to be displayed or overlaid onto the workflow graphic 400 in response to a cursor hovering over or selection one of the dashboard elements 406. However, other selection tools may be used without departing from the scope of the technology.

Visually indicating the status of the tasks on the workflow graphic 400 may include color-coding at least some of the depictions of the tasks or their linking graphics, as earlier described herein. Similar to previous embodiments, the method or computer program may include providing a selectable date format view which filters the KPIs mapped from the EHR database based on a selected period of times or dates, as well as a selectable individual order view in which the KPIs mapped from the EHR database may be filtered to display only KPIs associated with a single patient or a single order. In addition to other information described above, the KPIs may include any of the following: completion or turnaround time for any of the tasks, documentation of critical task-related information, task outcomes, task statistics, task comparisons, and task projections or predictions.

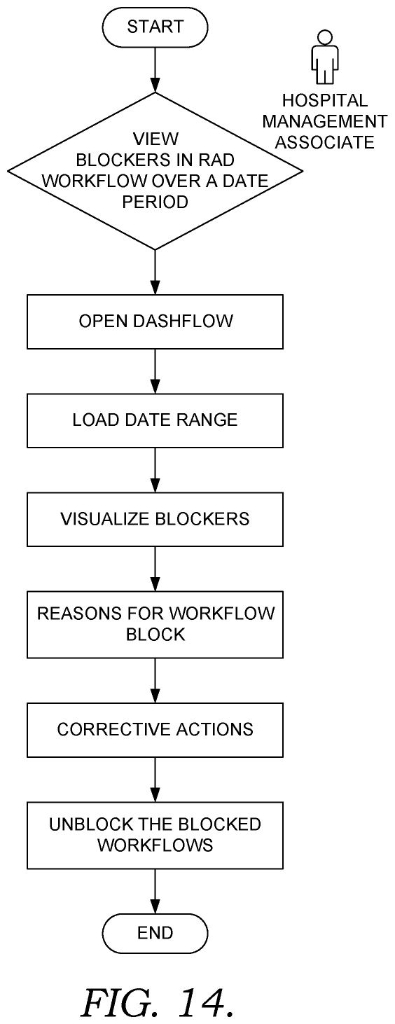

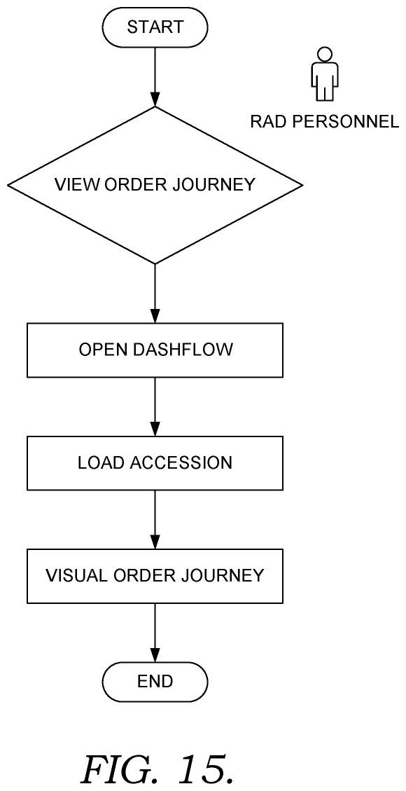

In use, the methods, computer programs, and systems described herein may be utilized by medical staff and management in a variety of ways. For example, as illustrated in FIG. 14, a hospital management associate may view and identify tasks causing a blocked workflow by first opening the interactive digital workflow graphic 400, selecting a date range, and viewing uncompleted or blocked tasks for one or more orders. Based on determining where such blockage is occurring, the hospital management associate may then investigate reasons for that workflow blockage, take corrective actions, and thereby unblock the blocked workflows. Similarly, as illustrated in FIG. 15, a radiology technician or other such personnel may use the interactive digital workflow graphic 400 to visualize an order on its journey, first by opening the workflow graphic 400, then loading a particular order, thereby visualizing the order's journey and how their tasks interact with other tasks therein.

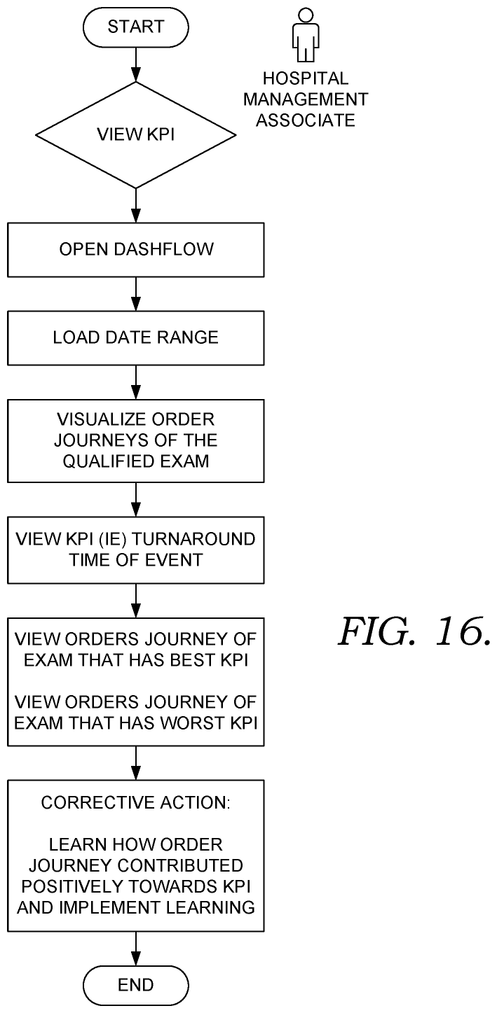

Finally, a hospital management associate may also use the technology herein to visualize KPIs for certain types of orders to determine ways to improve the workflow. For example, as depicted in FIG. 16, the hospital management associate may open the interactive digital workflow graphic 400 for a particular type of order, select a particular date range, and then visualize the order journeys of orders or exams that fall within that date range. Next, the hospital management associate may view the KPIs displayed therein, such as turnaround time of various tasks, as well as view overall KPIs such as which task has the best KPI (e.g., turnaround time) and which task in the workflow has the worst KPI (e.g., turnaround time). Finally, the hospital management associate may take corrective action by learning how the order journey contributed positively or negatively towards the KPIs displayed in the workflow and to implement corrective action based on lessons learned thereby.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described. Accordingly, the scope of the invention is intended to be limited only by the following claims.

What is claimed is:

1. One or more computer storage media having computer-executable instructions embodied thereon, that when executed, perform a method of displaying updated process flow data in a distributed system, the method comprising:
receiving a request for process flow data and key performance indicators (KPIs) for a particular order type within an electronic health record (EHR) database at a particular medical facility over a selected time period;
displaying a flow chart on a graphical user interface including depictions of a plurality of tasks associated with the particular order type and at least some of the plurality of tasks connected by linking graphics indicating a required or desired sequence of completion of the plurality of tasks;
visually indicating which of the plurality of tasks is completed, is in progress, or is queued for a pre-defined threshold number or percentage of orders of the particular order type within the selected time period; and
displaying on the flow chart, on or adjacent to at least one of the depictions of the plurality of tasks to be completed, dashboard elements for depicting the KPIs for at least one of the plurality of tasks displayed in the flow chart.

2. The one or more computer storage media of claim 1, wherein said KPIs are displayed automatically directly on the dashboard elements or are displayed or overlaid on the flow chart in response to a cursor hovering over or selecting one of the dashboard elements.

3. The one or more computer storage media of claim 1, wherein the visually indicating is accomplished via color-coding of at least some of the depictions of the plurality of tasks or the linking graphics.

4. The one or more computer storage media of claim 1, wherein the KPIs include statistics or data regarding at least one of completion or turnaround time for any of the plurality of tasks, documentation of critical task-related information, task outcomes, task statistics, task comparisons, and task projections or predictions.

5. The one or more computer storage media of claim 1, the method further comprising displaying additional linking graphics on the flow chart connecting additional tasks when one of a plurality of selectable order options are selected by a user.

6. The one or more computer storage media of claim 5, wherein the particular order type is a radiological exam, and the plurality of selectable order options include replacing an order, adding onto the order, vetting the order, or holding the order.

7. The one or more computer storage media of claim 1, the method further comprising providing a date selection element which filters the KPIs based on a selected period of time or dates entered into the date selection element.

8. The one or more computer storage media of claim 1, the method further comprising providing a selectable individual order view in which the KPIs are filtered to display only ones of the KPIs associated with a single patient or a single order of the particular order type.

9. A computer system comprising:
one or more processors on one or more computing devices programmed to:
build a workflow graphic configured for display on a graphical user interface, the workflow graphic including graphical depictions of a plurality of tasks to be completed for a particular medical order type, at least some of the plurality of tasks graphically connected by linking graphics indicating a required or desired completion sequence of the plurality of tasks, wherein the workflow graphic is built based on the particular medical order type, facilities or departments involved with the plurality of tasks for the particular medical order type, and sequences of the plurality of tasks within and between each of the facilities or departments for the particular medical order type;
map key performance indicators (KPIs) for one or more of the plurality of tasks from an electronic health record (EHR) database to one or more dashboard elements depicted on or adjacent to at least one of the graphical depictions of one of the plurality of tasks; and
visually indicate which of the plurality of tasks is completed, is in progress, or is queued for a pre-defined threshold number or percentage of orders of the particular medical order type.

10. The computer system of claim 9, wherein said KPIs are mapped to be displayed directly on the one or more dashboard elements or are mapped to be displayed or overlaid onto the workflow graphic in response to a cursor hovering over or selecting one of the one or more dashboard elements.

11. The computer system of claim 9, wherein the visually indicating comprises color-coding at least some of the graphical depictions of the plurality of tasks or the linking graphics.

12. The computer system of claim 9, wherein the one or more processors on the one or more computing devices is further programmed to provide a selectable date format view of the workflow graphic which filters the KPIs mapped from the EHR database based on a selected period of time or dates.

13. The computer system of claim 9, wherein the one or more processors on the one or more computing devices are further programmed to provide a selectable individual order view in which the KPIs mapped from the EHR database are filtered to display only the KPIs associated with a single patient or a single order of the particular medical order type.

14. The computer system of claim 9, wherein the KPIs include at least one of completion or turnaround time for any of the plurality of tasks, documentation of critical task-related information, task outcomes, task statistics, task comparisons, and task projections or predictions.

15. A computerized method comprising:
building, with a computer system comprising one or more processors, an interactive digital workflow graphic configured for display on a graphical user interface, the interactive digital workflow graphic including graphical depictions of a plurality of tasks to be completed for a particular radiological order type, at least some of the plurality of tasks graphically connected by linking graphics indicating a required or desired completion sequence of the plurality of tasks, wherein the interactive digital workflow graphic is built based on the particular radiological order type, facilities or departments involved with the plurality of tasks for the particular radiological order type, and sequences of the plurality of tasks within and between each of the facilities or departments for the particular radiological order type;
mapping key performance indicators (KPIs) for one or more of the plurality of tasks from an electronic health record (EHR) database to one or more dashboard elements depicted on or adjacent to at least one of the graphical depictions of one of the plurality of tasks; and
visually indicating on the interactive digital workflow graphic which of the plurality of tasks is completed, is in progress, or is queued for a pre-defined threshold number or percentage of orders of the particular radiological order type by color-coding at least some of the graphical depictions of the plurality of tasks or the linking graphics.

16. The computerized method of claim 15, wherein said KPIs are mapped to be displayed directly on the one or more dashboard elements or are mapped to be displayed or overlaid onto the interactive digital workflow graphic in response to a cursor hovering over or selecting one of the one or more dashboard elements.

17. The computerized method of claim 15, further comprising providing a selectable date format view of the interactive digital workflow graphic which filters the KPIs mapped from the EHR database based on a selected period of time or dates.

18. The computerized method of claim 15, further comprising providing a selectable individual order view in which the KPIs mapped from the EHR database are filtered to display only the KPIs associated with a single patient or a single order of the particular radiological order type.

19. The computerized method of claim 15, wherein the KPIs include at least one of completion or turnaround time for any of the plurality of tasks, documentation of critical task-related information, task outcomes, task statistics, task comparisons, and task projections or predictions.

20. The computerized method of claim 15, further comprising displaying additional linking graphics on the interactive digital workflow graphic connecting additional tasks when one of a plurality of selectable order options are selected by a user, the plurality of selectable order options include replacing an order, adding onto the order, vetting the order, or holding the order.
